(19) United States
(12) Patent Application Publication
(10) Pub. No.: US 2018/0146932 A1
(43) Pub. Date: May 31, 2018

(54) OPERATION TABLE HAVING ROBOT ARM

(71) Applicant: MEDICAROID CORPORATION, Kobe-shi (JP)

(72) Inventor: Kazunori SUGA, Kobe-shi (JP)

(73) Assignee: MEDICAROID CORPORATION, Kobe-shi (JP)

(21) Appl. No.: 15/814,647

(22) Filed: Nov. 16, 2017

(30) Foreign Application Priority Data
Nov. 29, 2016 (JP) 2016-231658
Nov. 29, 2016 (JP) 2016-231661
Sep. 27, 2017 (JP) 2017-187108

(51) Int. Cl. A61B 6/04

(52) U.S. Cl. CPC A61B 6/0457; A61B 6/4458; A61B 6/0407

(57) ABSTRACT

An embodiment of an operation table may include a table for loading a patient; a base buried or fixed to a floor; and a robot arm, a first end of the robot arm supported by the base and a second end of the robot arm supporting the table. The robot arm may include a vertical joint rotatable about a rotational axis extending in a horizontal direction; and a joint activation mechanism that activates the vertical joint. The joint activation mechanism may include: a motor; a first speed reducer that reduces a speed of rotation transmitted from the motor to output slower rotation; and a second speed reducer that reduces a speed of the slower rotation transmitted from the first speed reducer to output slower rotation.

### CROSS-REFERENCE TO RELATED APPLICATION

This application claims priority from prior Japanese Patent Applications No. 2016-231658, filed on Nov. 29, 2016, entitled “ROBOTIC OPERATION TABLE”, No. 2016-231661, filed on Nov. 29, 2016, entitled “ROBOTIC OPERATION TABLE”, and No. 2017-187108, filed on Sep. 27, 2017, entitled “ROBOTIC OPERATION TABLE AND ROBOTIC TREATMENT TABLE”, the entire contents of which are incorporated herein by reference.

### BACKGROUND

One or more embodiments disclosed herein relate to an operation table and a treatment table configured to move a table by a robot arm.

The U.S. Patent Publication No. 2005/0234327 A1 discloses a patient positioning assembly which moves a table, loaded with a patient, by a robot arm and determines the position of the patient with respect to a radiation source. The robot arm disclosed in this publication document includes a joint having a reduction ratio of 200 (i.e., 1/200 of input rotation is output).

There has been a demand for an operation table which is capable of easily moving a table, loaded with a patient, with less chance to interfere with peripheral devices. Thus, the patient positioning assembly disclosed in the U.S. Patent Publication No. 2005/0234327 A1 may be applied to an operation table used in an operating room in order to move a table loaded with a patient. In such a configuration, the patient-loaded table is easily movable and less likely to interfere with peripheral devices, unlike the case where the operation table is movable by means of casters.

However, a large robot arm is used for such a patient positioning assembly as disclosed in the U.S. Patent Publication No. 2005/0234327 A1, because due to irradiation, the robot arm is operated in a circumstance where no one is around without the need to pay attention to nearby workers. Thus, the application of the robot arm disclosed in the U.S. Patent Publication No. 2005/0234327 A1 to the operation table may result in leaving only a narrow space around the operation table, and inhibit the movements of the workers during surgery.

In addition, the operation table is required to ensure a high safety performance level since there is a chance to move a patient who is given a general anesthesia and unconscious. For example, the table loaded with a patient is required not to make a sudden downward movement even if the brakes of the robot are broken in a situation where power supply to the robot is cut off due to a problem such as a power failure. However, the robot arm disclosed in the U.S. Patent Publication No. 2005/0234327 A1 cannot move down slowly in the situation described above because the joint of said robot arm has only a small reduction ratio, i.e., 200.

### SUMMARY

One or more embodiments disclosed herein are intended to provide an operation table which substantially prevents a table from making a sudden downward movement even in a situation where a brake is broken while power supply is stopped, and which ensures a high safety performance level. One or more embodiments disclosed herein are also intended to provide a robotic treatment table with improved safety performance level compared to known art.

An operation table according to one or more embodiments may include: a table for loading a patient; a base buried or fixed to a floor; and a robot arm, a first end of the robot arm supported by the base and a second end of the robot arm supporting the table, wherein the robot arm comprises: a vertical joint rotatable about a rotational axis extending in a horizontal direction; and a joint activation mechanism that activates the vertical joint, wherein the joint activation mechanism comprises: a motor; a first speed reducer that reduces a speed of rotation transmitted from the motor to output slower rotation; and a second speed reducer that reduces a speed of the slower rotation transmitted from the first speed reducer to output slower rotation.

An operation table according to one or more embodiments may include: a table for loading a patient; a base buried or fixed to a floor; and a robot arm, a first end of the robot arm supported by the base and a second end of the robot arm supporting the table, wherein the robot arm comprises: a plurality of joints; and a plurality of joint activation mechanisms for the joints, wherein each of the plurality of joint activation mechanisms comprises: a motor; a first speed reducer that reduces a speed of rotation transmitted from the motor; and a second speed reducer that reduces a speed of rotation transmitted from the first speed reducer and activates the joint.

A treatment table according to one or more embodiments may include: a table for loading a patient; a base installed below a floor; and a robot arm, a first end of the robot arm supported by the base via a slide joint and a second end of the robot arm supporting the table, wherein the slide joint is configured to slide the robot arm in the vertical direction with respect to the base, wherein the robot arm comprises: a vertical joint rotatable about a rotational axis extending in a horizontal direction; and a joint activation mechanism that activates the vertical joint, wherein the joint activation mechanism comprises: a motor; a first speed reducer that reduces a speed of rotation transmitted from the motor to output slower rotation; and a second speed reducer that reduces a speed of the slower rotation transmitted from the first speed reducer to output slower rotation.

A treatment table according to one or more embodiments may include: a table for loading a patient; a base installed below a floor; and a robot arm, a first end of the robot arm supported by the base via a slide joint and a second end of the robot arm supporting the table, wherein the slide joint is configured to slide the robot arm in the vertical direction with respect to the base, wherein the robot arm comprises: a plurality of joints; and a plurality of joint activation mechanisms for the respective joints, wherein each of the plurality of joint activation mechanisms comprising: a motor; a first speed reducer that reduces a speed of rotation transmitted from the motor; and a second speed reducer that reduces a speed of rotation transmitted from the first speed reducer and activates the joint.

### BRIEF DESCRIPTION OF THE DRAWINGS

FIG. 1 is a diagram schematically illustrating a perspective view of a robotic operation table according to a first embodiment;

FIG. 2 is a diagram illustrating a perspective view of an articulated robot arm of the robotic operation table according to the first embodiment;

FIG. 3 is a diagram illustrating a perspective view of a vertical articulated arm assembly of the robotic operation table according to the first embodiment;

FIG. 4 is a diagram schematically illustrating a vertical joint of the robotic operation table according to the first embodiment;

FIG. 5 is a diagram schematically illustrating a speed reducer of the robotic operation table according to first embodiment;

FIG. 6 is a diagram illustrating a perspective view, as viewed from the top, of a linear movement assembly of the robotic operation table according to the first embodiment;

FIG. 7 is a diagram illustrating a perspective view, as viewed from the bottom, of the linear movement assembly of the robotic operation table according to the first embodiment;

FIG. 8 is a diagram schematically illustrating a perspective view of a robotic operation table according to a second embodiment;

FIG. 9 is a diagram illustrating a perspective view of an articulated robot arm of the robotic operation table according to the second embodiment;

FIG. 10 is a diagram schematically illustrating joints of the robotic operation table according to the second embodiment;

FIG. 11 is a diagram schematically illustrating a horizontal joint of a robotic operation table according to a variation of the second embodiment; and

FIG. 12 is a diagram schematically illustrating a robotic treatment table according to a third embodiment.

### DETAILED DESCRIPTION

Examples of one or more embodiments will be described in detail below with reference to the drawings.

### First Embodiment

(Configuration of Robotic Operation Table)

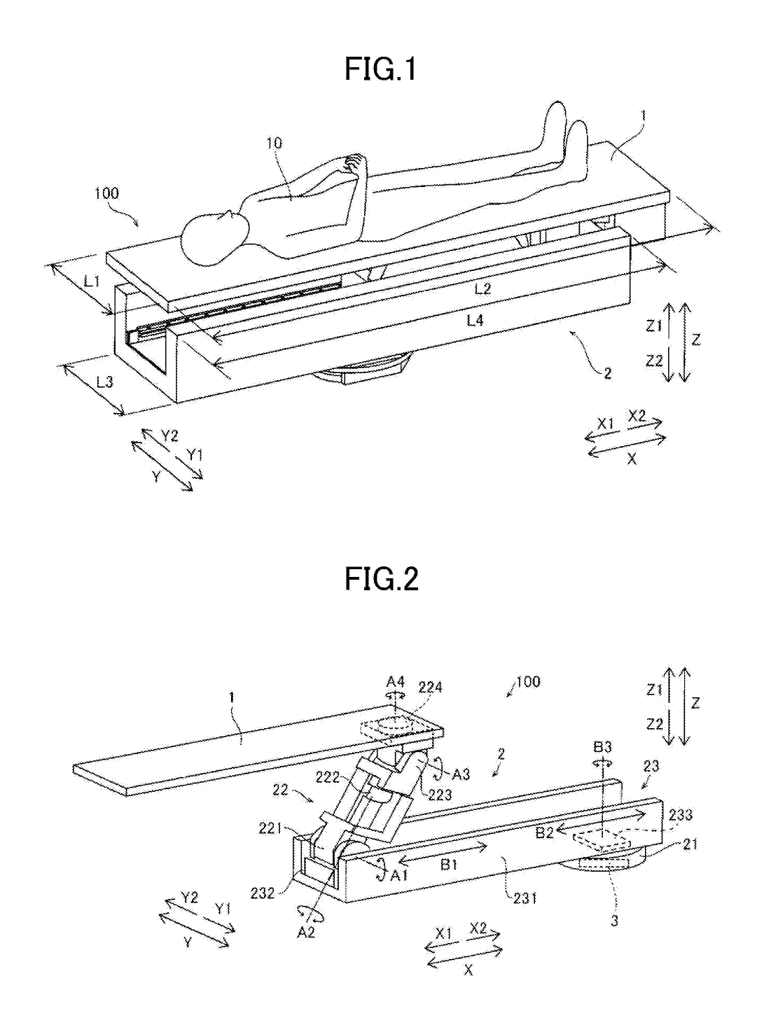

General configurations of a robotic operation table 100 according to a first embodiment will be described with reference to FIGS. 1 to 7.

The robotic operation table 100 has a table 1 for loading a patient, an articulated robot arm 2, and a controller 3 (see FIG. 2), as illustrated in FIG. 1. The articulated robot arm 2 has a base 21, a vertical articulated arm assembly 22, and a linear movement assembly 23 as illustrated in FIG. 2. The vertical articulated arm assembly 22 includes a vertical joint 221, a roll-rotational joint 222, a vertical joint 223, and a yaw-rotational joint 224. The linear movement assembly 23 includes a guide member 231, a sliding member 232, and a substrate member 233. Note that the vertical joint 221, the roll-rotational joint 222, the vertical joint 223, and the yaw-rotational joint 224 are examples of the “joint” or “joints” used in the claims.

The robotic operation table 100 may be used, for example, as an operation table for medical or surgical operations. The robotic operation table 100 may be movable to a loading position where a patient 10 is loaded on the table 1, and may be capable of moving the patient 10 lying on the table 1 to positions, such as an operation position, an inspection position, a treatment position, and an X-ray picture-taking position. The robotic operation table 100 may also be capable of tilting the patient 10 loaded on the table 1.

The table 1 has approximately a rectangular, flat plate-like shape. The top surface of the table 1 is approximately flat. The table 1 is moved by the articulated robot arm 2. Specifically, the table 1 is movable in a first direction (or an X direction) which is a horizontal direction, a second direction (or a Y direction) which is a horizontal direction orthogonal to the first direction, and a third direction (or a Z direction) which is a vertical direction orthogonal to the first and second directions. The table 1 can rotate about an axis extending in the X direction (i.e., a roll rotation). The table 1 can rotate about an axis extending in the Y direction (i.e., a pitch rotation). The table 1 can turn about an axis extending in the Z direction (i.e., a yaw turn).

The articulated robot arm 2 is configured to move the table 1. One end of the articulated robot arm 2 is supported on the base 21 fixed to a floor, and the other end of the articulated robot arm 2 supports the table 1 in a movable manner. The articulated robot arm 2 is supported on the base 21 such that the arm 2 can turn about the axis extending in the vertical direction (i.e., the Z direction), and the articulated robot arm 2 has six degrees of freedom to move the table 1. Specifically, the articulated robot arm 2 has four degrees of freedom achieved by the vertical articulated arm assembly 22. The four degrees of freedom include a rotation about a rotational axis A1, a rotation about a rotational axis A2, a rotation about a rotational axis A3, and a turn about a rotational axis A4. The articulated robot arm 2 has two degrees of freedom achieved by the linear movement assembly 23. The two degrees of freedom include linear movements along a B1 direction and a B2 direction and a turn about a rotational axis B3.

The articulated robot arm 2 is supported on the base 21 so as to turn about a turning axis extending in the Z direction. That is, the substrate member 233 is configured to turn about the turning axis B3 extending in the vertical direction (i.e., the Z direction) with respect to the base 21. The guide member 231 is configured to make a linear movement along the B2 direction with respect to the substrate member 233. The sliding member 232 is configured to make a linear movement along the B1 direction with respect to the guide member 231.

The vertical articulated arm assembly 22 is coupled to the guide member 231. The vertical joint 221 of the vertical articulated arm assembly 22 is configured to rotate about the rotational axis A1 extending in a direction approximately orthogonal to the moving direction (i.e., the B1 direction) of the guide member 231. The roll-rotational joint 222 is configured to rotate about the rotational axis A2 extending in a direction approximately orthogonal to the rotational axis of the vertical joint 221. The vertical joint 223 is configured to rotate about the rotational axis A3 extending in a direction approximately orthogonal to the rotational axis of the roll-rotational joint 222. The yaw-rotational joint 224 is configured to turn about the rotational axis A4 extending in a direction approximately orthogonal to the rotational axis of the vertical joint 223. That is, the vertical articulated arm assembly 22 is configured to have two vertical joints 221 and 223 and move the table 1 along multiple rotational degrees of freedom.

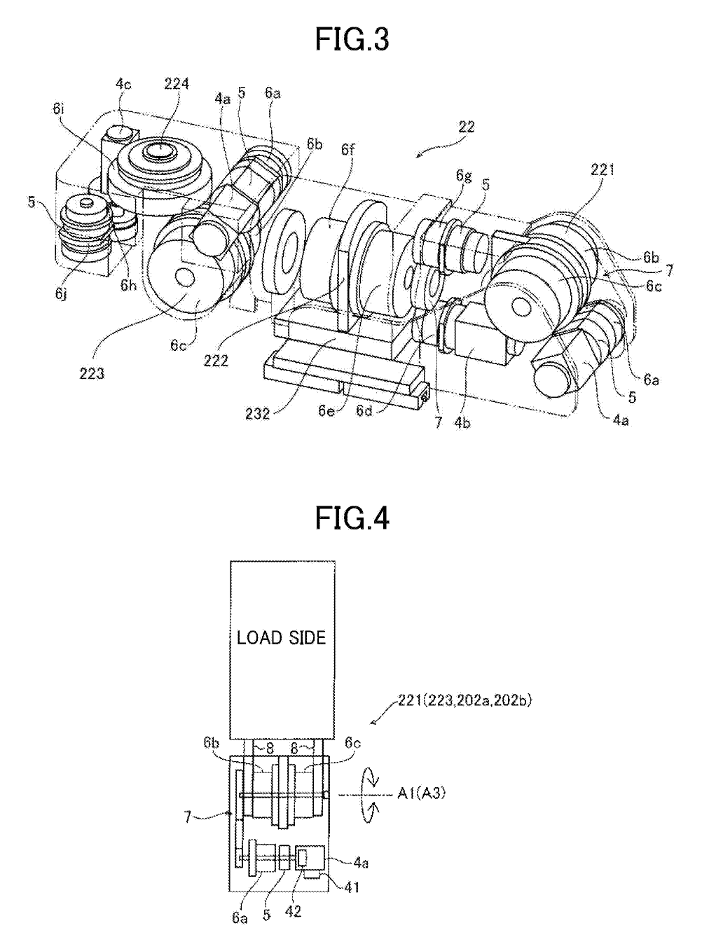

In the first embodiment, the vertical joint 221 (223) is rotatable about the rotational axis A1 (A3) extending in the horizontal direction. Further, as illustrated in FIGS. 3 and 4, an activation mechanism for the vertical joint 221 (223) has: a first motor 4a, which is a servomotor; a first speed reducer 6a which reduces a speed of rotation transmitted from the first motor 4a to output slower rotation; and second speed reducers 6b and 6c which reduce a speed of the slower rotation transmitted from the first speed reducer 6a to output slower rotation. The activation mechanism for the vertical joint 221 (223) also has a second electromagnetic brake 5, a gear portion 7, and connecting portions 8 as illustrated in FIG. 4.

In the first embodiment, the first motor 4a has an encoder 41 and a first electromagnetic brake 42 of a built-in type as illustrated in FIG. 4. The second electromagnetic brake 5 is attached to an output rotational shaft of the first motor 4a. The first electromagnetic brake 42 and the second electromagnetic brake 5 are configured to brake the vertical joint 221 (223). The encoder 41 is configured to detect a driving amount of the first motor 4a and transmit the detection result to the controller 3.

The first speed reducer 6a is configured as a wave gear reducer. The second speed reducers 6b and 6c are configured as wave gear reducers. The first speed reducer 6a and the second speed reducers 6b and 6c are connected in series. That is, two-stage reduction is realized by the first speed reducer 6a and the second speed reducers 6b and 6c. The second speed reducers 6b and 6c are connected in parallel. That is, the load received through the vertical joint 221 (223) is distributed and supported by the second speed reducers 6b and 6c.

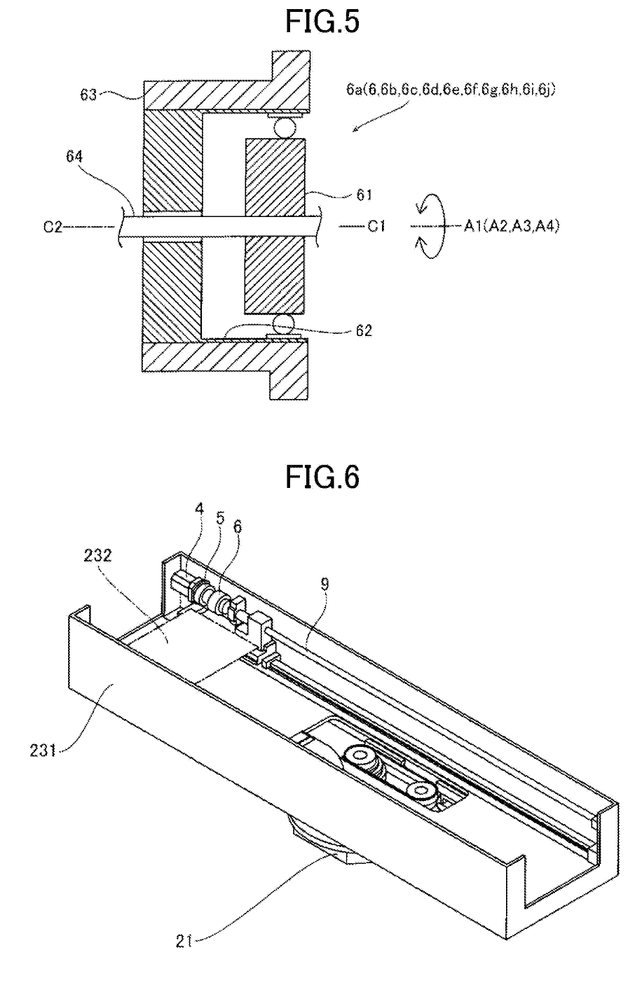

Each of the wave gear reducers includes, as illustrated in FIG. 5, an annular rigid internally-toothed gear 63, an annular flexible externally-toothed gear 62 arranged radially inside the rigid internally-toothed gear 63, and a wave generator 61. The wave generator 61 deforms the flexible externally-toothed gear 62 such that the externally-toothed gear 62 partially engages with the rigid internally-toothed gear 63 at two regions, and moves the engaging regions between the rigid internally-toothed gear 63 and the flexible externally-toothed gear 62 in a circumferential direction. The rigid internally-toothed gear 63 is fixedly provided. The wave generator 61 is attached to an input rotational shaft C1 of the wave gear reducer in a coaxial manner. The flexible externally-toothed gear 62 is coupled and fixed to an output rotational shaft C2 of the wave gear reducer. That is, in the wave gear reducer, the rotation is input to the wave generator 61, and the rotation is output at lower speed from the flexible externally-toothed gear 62.

In the first speed reducer 6a, the rotation of the first motor 4a is input to the wave generator 61 via the input rotational shaft 64. Then, the rotation is output at lower speed from the flexible externally-toothed gear 62 to the gear portion 7 via the shaft.

The input rotational shafts 64 of the second speed reducers 6b and 6c are coupled to each other in a coaxial manner, so that a plurality of second speed reducers 6b and 6c are arranged in parallel. Specifically, the second speed reducers 6b and 6c are arranged such that the respective wave generators 61 face inward to each other. The common input rotational shaft 64 is connected to the wave generators 61 to transmit rotation. Then, the rotation is output at lower speed from the flexible externally-toothed gears 62 located outward of the input rotational shaft 64. The output rotation is transmitted to the load side via the connecting portions 8. The vertical joint 221 (223) is driven in this manner.

The second speed reducers 6b and 6c have approximately the same reduction ratio. The output rotational shafts of the second speed reducers 6b and 6c are arranged on the rotational axis of the vertical joints 221 and 223 extending in the horizontal direction. The two second speed reducers 6b and 6c are installed such that their phases synchronize with each other in order that the load is evenly applied to the reducers 6b and 6c. Specifically, the engagement phases in which the wave generator 61, the flexible externally-toothed gear 62, and the rigid internally-toothed gear 63 are engaged with one another are synchronized in the two second speed reducers 6b and 6c. In this configuration, the relationship between torque and a twist angle is equal between the two second speed reducers 6b and 6c. This configuration allows the two second speed reducers 6b and 6c to output synchronized rotations, so that the distributed load can be transmitted via the connecting portions 8.

The total reduction ratio of the first speed reducer 6a and the second speed reducers 6b and 6c may be 1000 or more and 20000 or less. In the case where the reduction ratio is N, 1/N of input rotation is output. For example, in the case where the reduction ratio is 1000, the input rotational speed is reduced to output 1/1000 of the input rotation. The total reduction ratio of the first speed reducer 6a and the second speed reducers 6b and 6c may be 3000 or more and 10000 or less.

As illustrated in FIG. 3, an activation mechanism for the roll-rotational joint 222 has: a second motor 4b, which is a servomotor; a third speed reducer 6d which reduces a speed of rotation transmitted from the second motor 4b to output slower rotation; fourth speed reducers 6e and 6f which reduce a speed of the slower rotation transmitted from the third speed reducer 6d to output slower rotation; a speed reducer 6g which reduces the speed of the slower rotation transmitted from the third speed reducer 6d to output slower rotation; and a second electromagnetic brake 5 to which the slower rotation is transmitted from the speed reducer 6g. The second motor 4b has an encoder 41 and a first electromagnetic brake 42 of a built-in type.

The third speed reducer 6d is configured as a wave gear reducer. The fourth speed reducers 6e and 6f are configured as wave gear reducers. The speed reducer 6g is configured as a wave gear reducer. The third speed reducer 6d and the fourth speed reducers 6e and 6f are connected in series. That is, two-stage reduction is realized by the third speed reducer 6d and the fourth speed reducers 6e and 6f. The fourth speed reducers 6e and 6f are connected in parallel. That is, the load received through the roll-rotational joint 222 is distributed and supported by the fourth speed reducers 6e and 6f.

As illustrated in FIG. 3, the yaw-rotational joint 224 includes: a motor 4c, which is a servomotor; a speed reducer 6h which reduces a speed of rotation transmitted from the motor 4c to output slower rotation; a speed reducer 6i which reduces a speed of the slower rotation transmitted from the speed reducer 6h to output slower rotation; a speed reducer 6j which reduces the speed of the slower rotation transmitted from the speed reducer 6h to output slower rotation; and a second electromagnetic brake 5 to which the slower rotation is transmitted from the speed reducer 6j. The motor 4c has an encoder 41 and a first electromagnetic brake 42 of a built-in type.

The speed reducers 6h, 6i, and 6j are configured as wave gear reducers. The speed reducers 6h and 6i are connected in series. That is, two-stage reduction is realized by the speed reducers 6h and 6i.

As illustrated in FIG. 6, in order to move the sliding member 232 linearly with respect to the guide member 231, the linear movement assembly 23 includes: a motor 4, which is a servomotor; a second electromagnetic brake 5; a speed reducer 6 which reduces a speed of rotation transmitted from the motor 4 to output slower rotation; and a ball screw shaft 9 which reduces a speed of the slower rotation transmitted from the speed reducer 6 to output a slower linear movement. The sliding member 232 is screwed to the ball screw shaft 9, and the rotation of the ball screw shaft 9 causes the sliding member 232 to move along the guide member 231. The motor 4 has an encoder and a first electromagnetic brake of a built-in type.

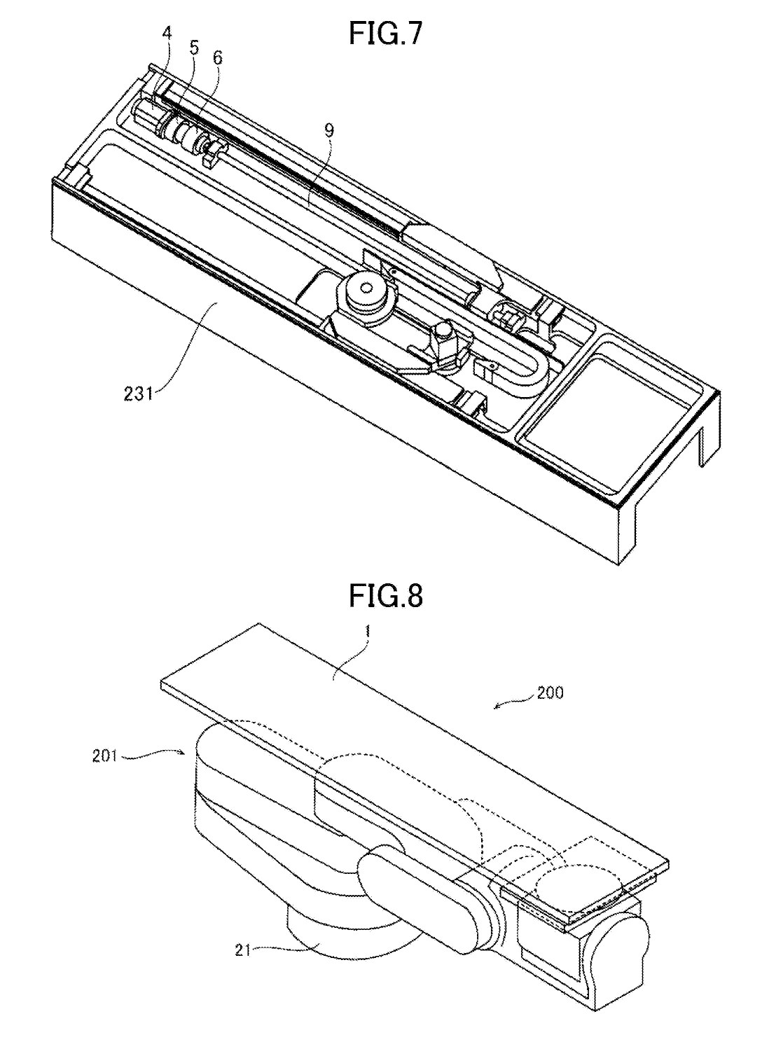

As illustrated in FIG. 7, in order to move the guide member 231 linearly with respect to the base 21, the linear movement assembly 23 has: a motor 4, which is a servomotor; a second electromagnetic brake 5; a speed reducer 6 which reduces a speed of rotation transmitted from the motor 4 to output slower rotation; and a ball screw shaft 9 which reduces a speed of the slower rotation transmitted from the speed reducer 6 to output a slower linear movement. The base 21 is screwed to the ball screw shaft 9, and the rotation of the ball screw shaft 9 causes the guide member 231 to move linearly with respect to the base 21. The motor 4 has an encoder and a first electromagnetic brake of a built-in type.

Further, the articulated robot arm 2 is configured to take a stored position while the table 1 is positioned at an operation position. While in the stored position, the articulated robot arm 2 is positioned in a storage space under the table 1. That is, the articulated robot arm 2 is folded and completely hidden under the table 1 as viewed from above (i.e., as viewed in the Z direction) when the table 1 is moved to a position where the patient 10 loaded on the table 1 undergoes an operation or a treatment. Note that the operation position is an example of the “predetermined position” used in the claims.

Specifically, as illustrated in FIG. 1, the articulated robot arm 2 in the stored position has a length L4 shorter than or equal to the length L2 of the table 1 in the first direction (i.e., the X direction), and a length L3 shorter than or equal to the length L1 of the table 1 in the second direction (i.e., the Y direction) orthogonal to the first direction.

The controller 3 is positioned in the base 21 and controls the movement of the table 1 by the articulated robot arm 2. Specifically, the controller 3 is configured to move the table 1 by controlling the activation of the articulated robot arm 2 based on an operation performed by medical personnel (i.e., an operator). The controller 3 has one or a plurality of processors comprised, for example, of a central processing unit (a CPU), and a memory including a read only memory (a ROM), a random access memory (a RAM), a memory device, etc. Examples of the memory device include a hard disk drive and a semiconductor memory.

### Advantages of First Embodiment

The following advantages may be obtained in the first embodiment.

In the first embodiment, as described above, the activation mechanism for each of the vertical joints 221 and 223 is provided with the first speed reducer 6a which reduces a speed of rotation transmitted from the first motor 4a to output slower rotation, and the second speed reducers 6b and 6c which reduce a speed of the slower rotation transmitted from the first speed reducer 6a to output slower rotation. Due to this configuration, two-stage reduction may be realized by the first speed reducer 6a and the second speed reducers 6b and 6c. Thus, the speed of activation of the vertical joints 221 and 223 may be slowed down. As a result, the speed of movement of the table 1 on which the patient 10 is loaded may be slowed down. Further, even if power supply to the articulated robot arm 2 is stopped in a circumstance where the electromagnetic brakes are broken, the two-stage reduction achieved by the first speed reducer 6a and the second speed reducers 6b and 6c may prevent the table 1 on which the patient 10 is loaded from making a sudden downward movement. As a result, the patient 10 may be moved at low speed, and the table 1 may be prevented from making a sudden downward movement even in a situation where the electromagnetic brakes are broken while power supply is stopped. Moreover, the two-stage reduction achieved by the first speed reducer 6a and the second speed reducers 6b and 6c may provide high output torque. The maximum output of the first motor 4a may thus be reduced and the first motor 4a may be downsized. The vertical joints 221 and 223 may be downsized accordingly, which contributes to downsizing of the articulated robot arm 2, while ensuring the torque of the vertical joints 221 and 223 of the articulated robot arm 2 which moves the table 1 for loading the patient 10 who will be undergoing surgery.

Further, in the first embodiment, the first motor 4a has the first electromagnetic brake 42 of a built-in type, and the second electromagnetic brake 5 is attached to the output rotational shaft of the first motor 4a as described above. In addition to the two-stage speed reduction, this configuration allows for applying brakes to the vertical joints 221 and 223 by the two-stage braking system achieved by the first electromagnetic brake 42 and the second electromagnetic brake 5. Thus, a sudden downward movement of the table 1 may be more reliably prevented even if one of these brakes is broken while power supply is stopped.

In the first embodiment, the articulated robot arm 2 is supported on the base 21 such that the arm 2 can turn about the axis extending in the vertical direction (i.e., the Z direction), and the articulated robot arm 2 has six or more degrees of freedom to move the table 1 as described above. Having six or more degrees of freedom, the articulated robot arm 2 may easily move the table 1 to a desired position.

In the first embodiment, the articulated robot arm 2 includes the vertical articulated arm assembly 22 which has two or more vertical joints 221 and 223 and moves the table 1 along a multiple degrees of freedom, as described above. Since the articulated robot arm 2 may be provided with a plurality of vertical joints 221 and 223 which may be activated at low speed, the table 1 may be easily moved to a desired position in the vertical direction. In addition, a sudden downward movement of the table 1 may be effectively prevented even if the electromagnetic brakes are broken while power supply is stopped.

In the first embodiment, as described above, the vertical articulated arm assembly 22 is provided with the roll-rotational joint 222. The activation mechanism for the roll-rotational joint 222 is provided with the second motor 4b, the third speed reducer 6d which reduces a speed of rotation transmitted from the second motor 4b to output slower rotation, and the fourth speed reducers 6e and 6f which reduce a speed of the slower rotation transmitted from the third speed reducer 6d to output slower rotation. This configuration allows the patient 10 to be moved at low speed in a roll rotation of the table 1.

In the first embodiment, as described above, the articulated robot arm 2 includes the linear movement assembly 23 which moves the table 1 along a horizontal, linear degree of freedom. The linear movement assembly 23 allows the table 1 to be easily moved to a desired position in the horizontal direction.

In the first embodiment, as described above, the articulated robot arm 2 is configured to take the stored position, in which the articulated robot arm 2 is positioned in a storage space under the table 1, while the table 1 is positioned at the operation position. Due to this configuration, the articulated robot arm 2 may be less likely to interfere with the medical personnel during an operation.

In the first embodiment, as described above, the articulated robot arm 2 in the stored position has a length shorter than or equal to the length of the table 1 as viewed in the first direction (i.e., the X direction), and a length shorter than or equal to the length of the table 1 as viewed in the second direction (i.e., the Y direction). This configuration prevents the articulated robot arm 2 from protruding from the table 1 during medical practice, such as an operation. Thus, the articulated robot arm 2 may be less likely to interfere with the medical personnel, such as a surgeon, an assistant, a nurse, and a medical technologist.

In the first embodiment, the first speed reducer 6a and the second speed reducers 6b and 6c are configured as wave gear reducers as described above. The wave gear reducers may downsize the first speed reducer 6a and the second speed reducers 6b and 6c and may achieve an effective speed reduction.

In the first embodiment, as described above, each of the wave gear reducers includes a circular rigid internally-toothed gear 63, a circular flexible externally-toothed gear 62 arranged radially inside the rigid internally-toothed gear 63, and a wave generator 61 which deforms the flexible externally-toothed gear 62 such that the externally-toothed gear 62 partially engages with the rigid internally-toothed gear 63 at two regions, and moves the engaging regions between the rigid internally-toothed gear 63 and the flexible externally-toothed gear 62 in the circumferential direction. The rigid internally-toothed gear 63 is fixedly arranged. The wave generator 61 is attached to the input rotational shaft of the wave gear reducer in a coaxial manner. The flexible externally-toothed gear 62 is coupled and fixed to the output rotational shaft of the wave gear reducer. Having the rigid internally-toothed gear 63, the flexible externally-toothed gear 62, and the wave generator 61, the wave gear reducer may achieve the deceleration easily and stably.

In the first embodiment, as described above, the input rotational shafts of the second speed reducers 6b and 6c of the respective vertical joints 221 and 223 are coupled to each other in a coaxial manner, so that a plurality of second speed reducers 6b and 6c are arranged in parallel. The plurality of second speed reducers 6b and 6c have approximately the same reduction ratio. The output rotational shafts of the second speed reducers 6b and 6c are arranged on the rotational axis of the vertical joints 221 and 223 extending in the horizontal direction. This configuration allows the load to be distributed and supported by the plurality of second speed reducers 6b and 6c arranged in parallel. As a result, the resistance to load of the vertical joints 221 and 223 may be easily increased.

In the first embodiment, the total reduction ratio of the first speed reducer 6a and the second speed reducers 6b and 6c is set to be 1000 or more and 20000 or less as described above. As a result, compared to the case where the reduction ratio is less than 1000, the patient 10 may be moved at lower speed, and the table 1 may be less likely to make a sudden downward movement even in a situation where the electromagnetic brakes are broken while power supply is stopped. Further, compared to the case where the reduction ratio is greater than 20000, the vertical joints 221 and 223 may be less likely to be activated at excessively low speed.

In the first embodiment, the total reduction ratio of the first speed reducer 6a and the second speed reducers 6b and 6c is set to be 3000 or more and 10000 or less as described above. As a result, the patient 10 may be moved at low speed with reliability, and the table 1 may be effectively prevented from making a sudden downward movement even in a situation where the electromagnetic brakes are broken while power supply is stopped. Further, the vertical joints 221 and 223 may be effectively prevented from being activated at excessively low speed.

### Second Embodiment

Now, a second embodiment of one or more embodiments disclosed herein will be described with reference to FIGS. 8 to 10. Unlike the first embodiment illustrating the articulated robot arm including a linear movement assembly, an example in which the articulated robot arm includes a horizontal articulated arm assembly will be described in the second embodiment. Note that similar reference characters are used to designate elements similar to those of the first embodiment.

(Configuration of Robotic Operation Table)

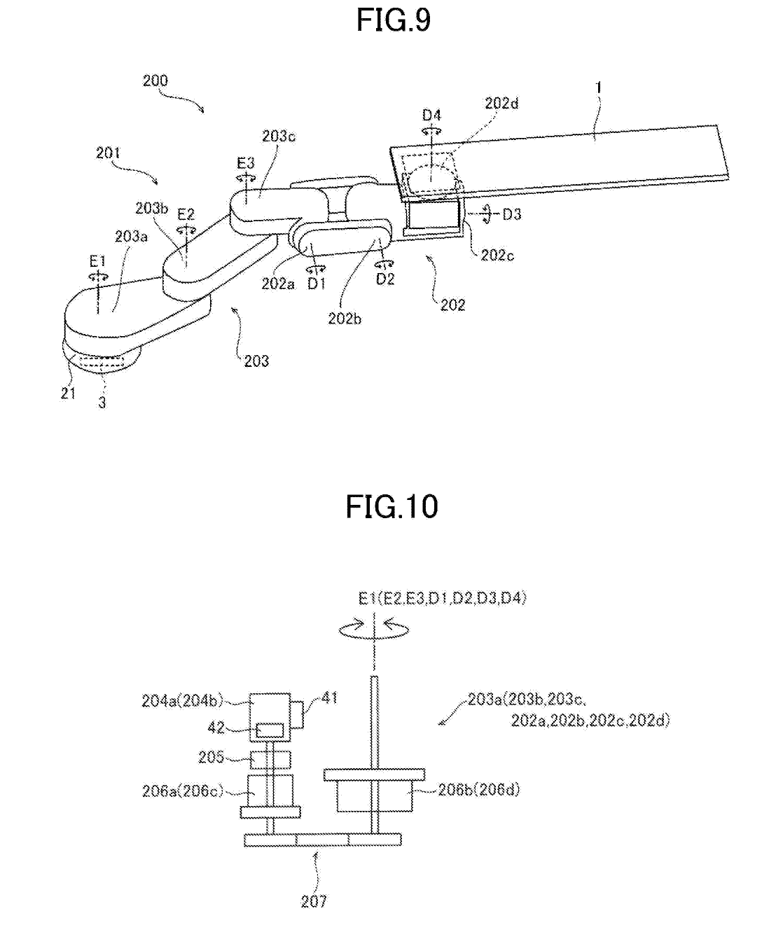

As illustrated in FIG. 8, a robotic operation table 200 has a table 1 for loading a patient, an articulated robot arm 201, and a controller 3 (see FIG. 9). As illustrated in FIG. 9, the articulated robot arm 201 has a base 21, a vertical articulated arm assembly 202, and a horizontal articulated arm assembly 203. The vertical articulated arm assembly 202 includes vertical joints 202a and 202b, a roll-rotational joint 202c, and a yaw-rotational joint 202d. The horizontal articulated arm assembly 203 includes horizontal joints 203a, 203b, and 203c. Note that the vertical joints 202a and 202b, the roll-rotational joint 202c, the yaw-rotational joint 202d, and the horizontal joints 203a, 203b, and 203c are examples of the “joint” or “joints” used in the claims.

The articulated robot arm 201 is configured to move the table 1. One end of the articulated robot arm 201 is supported on the base 21 fixed to a floor, and the other end of the articulated robot arm 201 supports the table 1 in a movable manner. The articulated robot arm 201 is configured to move the table 1 along seven degrees of freedom. Specifically, the articulated robot arm 201 has four degrees of freedom achieved by the vertical articulated arm assembly 202. The four degrees of freedom include a rotation about a rotational axis D1, a rotation about a rotational axis D2, a rotation about a rotational axis D3, and a turn about a rotational axis D4. The articulated robot arm 201 also has three degrees of freedom achieved by the horizontal articulated arm assembly 203. The three degrees of freedom include a turn about a rotational axis E1, a turn about a rotational axis E2, and a turn about a rotational axis E3.

In the second embodiment, the vertical joint 202a (202b) is rotatable about the rotational axis D1 (D3) extending in the horizontal direction. Further, as illustrated in FIG. 10, the vertical joint 202a (202b) has: a first motor 204a; a first speed reducer 206a which reduces a speed of rotation transmitted from the first motor 204a to output slower rotation; and a second speed reducer 206b which reduces a speed of the slower rotation transmitted from the first speed reducer 206a to output slower rotation. The vertical joint 202a (202b) also has a second electromagnetic brake 205 and a gear portion 207.

Similarly to the vertical joint 202a (202b), each of the horizontal joints 203a, 203b, and 203c, and the yaw-rotational joint 202d has a first motor 204a, a second electromagnetic brake 205, a first speed reducer 206a, a second speed reducer 206b, and a gear portion 207. Further, the roll-rotational joint 202c has a second motor 204b, a second electromagnetic brake 205, a third speed reducer 206c, a fourth speed reducer 206d, and a gear portion 207.

In the second embodiment, the first motor 204a (the second motor 204b) has an encoder 41 and a first electromagnetic brake 42 of a built-in type as illustrated in FIG. 10. The second electromagnetic brake 205 is attached to an output rotational shaft of the first motor 204a (the second motor 204b). The first and second electromagnetic brakes 42 and 205 are configured to brake the joints (i.e., the vertical joints 202a and 202b, the roll-rotational joint 202c, the yaw-rotational joint 202d, and the horizontal joints 203a, 203b, and 203c). The encoder 41 is configured to detect a driving amount of the first motor 204a and transmit the detection result to the controller 3.

The other configurations of the second embodiment are the same as, or similar to, those of the first embodiment.

### Advantages of Second Embodiment

The following advantages may be obtained in the second embodiment.

Similarly to the first embodiment, each of the vertical joints 202a and 202b in the second embodiment is provided with, as described above, the first speed reducer 206a which reduces a speed of rotation transmitted from the first motor 204a to output slower rotation, and the second speed reducers 206b which reduces a speed of the slower rotation transmitted from the first speed reducer 206a to output slower rotation. As a result, the patient 10 may be moved at low speed, and the table 1 may be prevented from making a sudden downward movement even when power supply is stopped.

In the second embodiment, the articulated robot arm 201 is configured to move the table 1 along seven or more degrees of freedom as described above. Having seven or more degrees of freedom, the articulated robot arm 201 may easily move the table 1 to a desired position.

In the second embodiment, as described above, the articulated robot arm 201 includes the horizontal articulated arm assembly 203 which moves the table 1 along multiple rotational degrees of freedom. Thus, the horizontal articulated arm assembly 203 allows the table 1 to be easily moved to a desired position in the horizontal direction.

The other advantages of the second embodiment are the same as, or similar to, those of the first embodiment.

### Variations of Second Embodiment

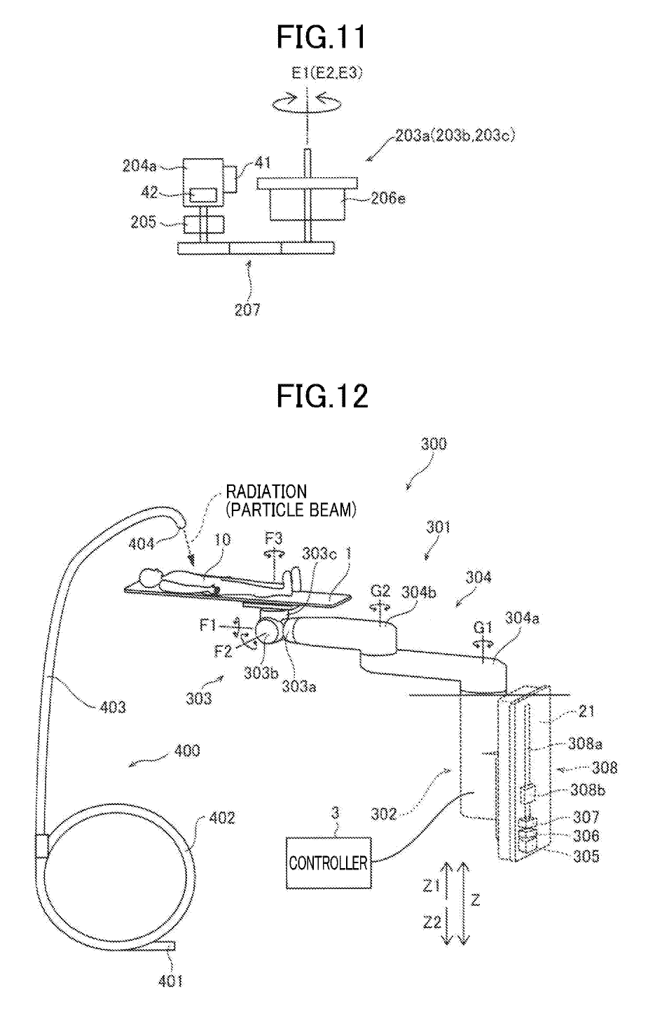

A single speed reducer 206e may be provided for each of the joints of the horizontal articulated arm assembly 203 of the articulated robot arm 201 according to the second embodiment. That is, each joint of the horizontal articulated arm assembly 203 may be decelerated by the single speed reducer 206e, and each joint of the vertical articulated arm assembly 202 may be decelerated by the two speed reducers, i.e., the first speed reducer 206a (the third speed reducer 206c) and the second speed reducer 206b (the fourth speed reducer 206d). Specifically, each of the horizontal joints 203a, 203b and 203c is provided with the single speed reducer 206e, as illustrated in FIG. 11. Each of the vertical joint 202a and 202b and the yaw-rotational joint 202d is provided with the first speed reducer 206a and the second speed reducer 206b, as illustrated in 10. The roll-rotational joint 202c is provided with the third speed reducer 206c and the forth speed reducer 206d, as illustrated in FIG. 10.

### Third Embodiment

Now, a third embodiment of one or more embodiments disclosed herein will be described with reference to FIG. 12. In the third embodiment, an example configuration of a robotic treatment table which includes a table and an articulated robot arm will be described. Note that similar reference characters are used to designate elements similar to those of the first embodiment.

(Configuration of Robotic Treatment Table)

As illustrated in FIG. 12, a robotic treatment table 300 includes a table 1 for loading a patient, an articulated robot arm 301, and a controller 3. The articulated robot arm 301 has a sliding joint 302, a vertical articulated arm assembly 303, and a horizontal articulated arm assembly 304. The sliding joint 302 includes a motor 305, an electromagnetic brake 306, a speed reducer 307, and a ball screw mechanism 308. The vertical articulated arm assembly 303 includes vertical joints 303a and 303b and a yaw-rotational joint 303c. The horizontal articulated arm assembly 304 includes horizontal joints 304a and 304b. Note that the vertical joints 303a and 303b, the yaw-rotational joint 303c, and the horizontal joints 304a and 304b are examples of the “joint” or “joints” used in the claims.

The articulated robot arm 301 is configured to move the table 1. One end of the articulated robot arm 301 is supported on the base 21 buried in a floor via the sliding joint 302, and the other end of the articulated robot arm 301 supports the table 1 in a movable manner. The articulated robot arm 301 is configured to move the table 1 along six degrees of freedom. Specifically, the articulated robot arm 301 has a vertical, linear degree of freedom achieved by the sliding joint 302. The articulated robot arm 301 also has three degrees of freedom achieved by the vertical articulated arm assembly 303. The three degrees of freedom include a rotation about a rotational axis F1, a rotation about a rotational axis F2, and a turn about a rotational axis F3. The articulated robot arm 301 also has two degrees of freedom achieved by the horizontal articulated arm assembly 304. The two degrees of freedom include a turn about a rotational axis G1 and a turn about a rotational axis G2.

In the third embodiment, the vertical joint 303a (303b) is rotatable about the rotational axis F1 (F2) extending in the horizontal direction. Further, as illustrated in FIG. 10, the vertical joint 303a (303b) has: a first motor 204a; a first speed reducer 206a which reduces a speed of rotation transmitted from the first motor 204a to output slower rotation; and a second speed reducer 206b which reduces a speed of the slower rotation transmitted from the first speed reducer 206a to output slower rotation. The vertical joint 303a (303b) also has a second electromagnetic brake 205 and a gear portion 207.

Similarly to the vertical joint 303a (303b), each of the horizontal joints 304a and 304b and the yaw-rotational joint 303c has a first motor 204a, a second electromagnetic brake 205, a first speed reducer 206a, a second speed reducer 206b, and a gear portion 207, as illustrated in FIG. 10.

In the third embodiment, the first motor 204a has an encoder 41 and a first electromagnetic brake 42 of a built-in type as illustrated in FIG. 10. The second electromagnetic brake 205 is attached to an output rotational shaft of the first motor 204a. The first and second electromagnetic brakes 42 and 205 are configured to brake the joints (i.e., the vertical joints 303a and 303b, the yaw-rotational joint 303c, and the horizontal joints 304a and 304b). The encoder 41 is configured to detect a driving amount of the first motor 204a and transmit the detection result to the controller 3.

The first speed reducer 206a is configured as a planetary gear reducer. The second speed reducer 206b is configured as an eccentric oscillation planetary gear reducer (an RV reducer). The first speed reducer 206a and the second speed reducer 206b are connected in series. That is, two-stage reduction is realized by the first and second speed reducers 206a and 206b.

The eccentric oscillation planetary gear reducer includes a first-stage reducer and a second-stage reducer. The first-stage reducer includes an input gear and a spur gear having a greater number of teeth than the input gear. The second-stage reducer includes: a rotational shaft having an eccentric portion and coupled to the spur gear; an internally-toothed gear; and an externally-toothed planetary gear which engages with the eccentric portion and comes into contact with the internally-toothed gear from radially inside, so that the externally-toothed planetary gear rotates eccentrically while meshing with different portions of the internally-toothed gear.

The articulated robot arm 301 is movable along the vertical, linear degree of freedom achieved by the sliding joint 302. Specifically, the articulated robot arm 301 is supported on the base 21 via the sliding joint 302. As illustrated in FIG. 12, the sliding joint 302 has: the motor 305, which is a servomotor; the electromagnetic brake 306; the speed reducer 307 which reduces a speed of rotation transmitted from the motor 305 to output slower rotation; and the ball screw shaft 308a which reduces a speed of the slower rotation transmitted from the speed reducer 307 to output a slower linear movement. A sliding member 308a is screwed to the ball screw shaft 308a, and the rotation of the ball screw shaft 308a causes the sliding member 308b to move along a guide member. Along with the movement of the sliding member 308b, the articulated robot arm 301 connected to the sliding member 308b moves along the vertical direction. The motor 305 has an encoder and a first electromagnetic brake of a built-in type.

The sliding joint 302 is provided below the floor surface. That is, the base 21 is fixed at a position below the floor surface.

The robotic treatment table 300 is used for positioning a patient in a radiation treatment system. Radiation includes X-rays, gamma rays, an electron beam, and a particle beam. The particle beam includes accelerated nuclei. The nuclei of the particle beam include hydrogen nuclei (i.e., protons), helium nuclei, carbon nuclei (i.e., carbon ions), neon nuclei, silicon nuclei, and argon nuclei. In a case of a treatment system using the particle beam, the robotic treatment table 300 is used as a table for positioning a patient in such a system.

The robotic treatment table 300 is used for adjusting an irradiation position where the patient 10 is irradiated with a particle beam (i.e., radiation) emitted from a particle beam irradiation device 400. The patient 10 is accurately positioned so that a treatment site of the patient 10 can be accurately irradiated with the particle beam. The robotic treatment table 300 is used to position the table 1 (i.e., the patient 10) accurately in three dimensions by the movement of the articulated robot arm 301. The particle beam irradiation device 400 has an entrance portion 401, an accelerator portion 402, a transport portion 403, and an emitting portion 404.

The entrance portion 401 is configured to generate a particle beam and lead the particle beam into the accelerator portion 402. Specifically, ions are generated by an ion source at the entrance portion 401. These ions are accelerated by a linear accelerator and delivered to the accelerator portion 402. The accelerator portion 402 is configured to generate a magnetic field and accelerate the particle beam entered. The accelerator portion 402 has an annular shape so that the particle beam is accelerated while passing through the annular accelerator portion 402.

The transport portion 403 is configured to transport the accelerated particle beam. The emitting portion 404 is configured to scan and irradiate the treatment site of the patient 10 with the particle beam. The emitting portion 404 may be configured to scatter, and thereby shape, the particle beam, and apply the thus obtained particle beam to the patient 10.

The other configurations of the third embodiment are the same as, or similar to, those of the first embodiment.

### Advantages of Third Embodiment

The following advantages may be obtained in the third embodiment.

As described above, according to the third embodiment, each of the vertical joints 303a and 303b is provided with the first speed reducer 206a which reduces a speed of rotation transmitted from the first motor 204a to output slower rotation, and the second speed reducer 206b which reduces a speed of the slower rotation transmitted from the first speed reducer 206a to output slower rotation. As a result, the patient 10 may be moved at low speed, and the table 1 may be prevented from making a sudden downward movement even when power supply is stopped. It is therefore possible to provide the robotic treatment table 300 with improved safety performance level compared to known art.

According to the third embodiment, the robotic treatment table 300 is used for positioning a patient in a particle beam treatment system, as described above. This configuration allows the patient 10 to be positioned accurately by the robotic treatment table 300 in the particle beam treatment. The affected area of the patient 10 can thus be irradiated with the particle beam with accuracy.

According to the third embodiment, the sliding joint 302 includes the motor 305, the speed reducer 307, and the ball screw mechanism 308, as described above. As a result, two-stage reduction may be achieved by the speed reducer 307 and the ball screw mechanism 308. It is therefore possible to effectively prevent the table 1 from making a sudden downward movement even in a situation where the electromagnetic brakes are broken while power supply is stopped.

### Variations

The embodiments disclosed herein are meant to be illustrative in all respects and should not be construed to be limiting in any manner. The scope of one or more embodiments of an operation table having a robot arm is defined not by the above-described embodiments, but by the scope of claims, and includes all modifications within equivalent meaning and scope to those of the claims.

For example, an example in which wave gear reducers are used as the first and second speed reducers has been described in the first and second embodiments, and an example in which a planetary gear reducer is used as the first speed reducer, and an eccentric oscillation planetary gear reducer is used as the second speed reducer has been described in the third embodiment. However, these are non-limiting examples. For example, in one or more embodiments, the first and second speed reducers may be any combination of the wave gear reducer, the planetary gear reducer, and the eccentric oscillation planetary gear reducer. Speed reducers other than the wave gear reducer, the planetary gear reducer, and the eccentric oscillation planetary gear reducer may also be used for the first and second speed reducers.

An example in which wave gear reducers are used for both of the first and second speed reducers has been described in the first and second embodiments, but this is a non-limiting example. In one or more embodiments, the wave gear reducer, the planetary gear reducer, or the eccentric oscillation planetary gear reducer may be used for at least one of the first or second speed reducer.

An example in which the articulated robot arm has six degrees of freedom has been described in the first and third embodiments, and an example in which the articulated robot arm has seven degrees of freedom has been described in the second embodiment, but these are non-limiting examples. In one or more embodiments, the robot arm may have five or less degrees of freedom, or eight or more degrees of freedom.

An example in which the articulated robot arm has two vertical joints has been described in the first to third embodiments, but this is a non-limiting example. In one or more embodiments, the articulated robot arm may have three or more vertical joints, or a single vertical joint.

An example in which two second speed reducers are arranged in parallel in a single joint has been described in the first embodiment, but this is a non-limiting example. In one or more embodiments, a single second speed reducer, or three or more second speed reducers may be provided in a single joint.

An example in which the base is fixed to a floor has been described in the first and second embodiments, but this is a non-limiting example. In one or more embodiments, the base may be buried and fixed in the floor.

An example in which the rotational axis of the motor and the rotational axis of the joint are approximately parallel to each other has been described in the first to third embodiments, but this is a non-limiting example. In one or more embodiments, the rotational axis of the motor and the rotational axis of the joint do not need to be parallel to each other. For example, a gear may be used to change the direction of the rotational axis of the joint to a direction intersecting with the rotational axis of the motor. In such a case, the gear used to change the direction of the rotational axis of the joint may be integrally formed with the speed reducer.

An example in which the sliding joint includes a ball screw mechanism has been described in the third embodiment, but this is a non-limiting example. In one or more embodiments, the sliding joint may include a rack and pinion mechanism.

An example in which the robotic treatment table is used in a particle beam treatment system using a particle beam for treatment has been described in the third embodiment, but this is a non-limiting example. The robotic treatment table may also be used in a radiation treatment system using radiation, other than the particle beam, for treatment.

An example in which the controller 3 is arranged in the base has been described in the first to third embodiments, but this is a non-limiting example. In one or more embodiments, the robot controller may be housed in a casing to serve as a control box. This control box may be placed, for example, at any location in an operating room or a treatment room, or may be placed in a different room from an operating room and a treatment room.

1. An operation table comprising:
a table for loading a patient;
a base buried or fixed to a floor; and
a robot arm, a first end of the robot arm supported by the base and a second end of the robot arm supporting the table, wherein the robot arm comprises:
a vertical joint rotatable about a rotational axis extending in a horizontal direction; and
a joint activation mechanism that activates the vertical joint, wherein the joint activation mechanism comprises:
a motor;
a first speed reducer that reduces a speed of rotation transmitted from the motor to output slower rotation; and
a second speed reducer that reduces a speed of the slower rotation transmitted from the first speed reducer to output slower rotation.

2. The operation table of claim 1, wherein the motor comprises a built-in electromagnetic brake.

3. The operation table of claim 1, wherein the first of the robot arm is supported by the base such that the robot arm rotates about an axis extending in a vertical direction.

4. The operation table of claim 1, wherein the robot arm has at least six degrees of freedom to move the table.

5. The operation table of claim 1, wherein the robot arm further comprises a first arm assembly comprising:
a plurality of the vertical joints; and
a plurality of the joint activation mechanisms for the vertical joints.

6. The operation table of claim 5, wherein the first arm assembly further comprises:
a roll joint rotatable about a roll axis; and
a roll joint activation mechanism that activates the roll joint, wherein
the roll joint activation mechanism comprises:
a second motor;
a third speed reducer that reduces a speed of rotation transmitted from the second motor to output slower rotation; and
a fourth speed reducer that reduces a speed of the slower rotation transmitted from the third speed reducer to output slower rotation.

7. The operation table of claim 5, wherein the robot arm further comprises a second arm assembly that supports the first arm assembly, comprising:
a plurality of horizontal joints; and
a plurality of horizontal joint activation mechanisms for the horizontal joints, wherein
each of the horizontal joints is rotatable about a rotational axis extending in a vertical direction.

8. The operation table of claim 7, wherein at least one of the second joint activation mechanisms comprises:
a second motor; and
a third speed reducer that reduces a speed of rotation transmitted from the second motor to output slower rotation.

9. The operation table of claim 7, wherein at least one of the second joint activation mechanisms comprises:
a second motor;
a third speed reducer that reduces a speed of rotation transmitted from the second motor to output slower rotation; and
a fourth speed reducer that reduces a speed of the slower rotation transmitted from the third speed reducer to output slower rotation.

10. The operation table of claim 1, wherein the robot arm is configured to take a stored posture, in which the robot arm is stored under the table, when the table is positioned at a predetermined position.

11. The operation table of claim 10, wherein the robot arm in the stored posture has a length shorter than or equal to a longitudinal length of the table in a horizontal state and a width shorter than or equal to a width of the table orthogonal to the longitudinal length.

12. The operation table of claim 1, wherein one of the first and second speed reducers is a wave gear reducer, a planetary gear reducer, or an eccentric oscillation planetary gear reducer.

13. The operation table of claim 1, wherein the joint activation mechanism comprising a third speed reducer including an input rotational shaft that is coupled to an input rotational shaft of the second speed reducer, the second and third speed reducers are arranged in parallel, and
the second and third speed reducers have approximately a same reduction ratio, and output rotational shafts of the second and third speed reducers are arranged on the rotational axis of the vertical joint.

14. The operation table of claim 1, wherein a total reduction ratio of the first and second speed reducers is 1000 or more and 20000 or less.

15. The operation table of claim 1, wherein the robot arm includes a linear movement assembly that moves the table along a linear degree of freedom.

16. An operation table comprising:
a table for loading a patient;
a base buried or fixed to a floor; and
a robot arm, a first end of the robot arm supported by the base and a second end of the robot arm supporting the table, wherein the robot arm comprises:
a plurality of joints; and
a plurality of joint activation mechanisms for the joints, wherein each of the plurality of joint activation mechanisms comprises:
a motor;
a first speed reducer that reduces a speed of rotation transmitted from the motor; and
a second speed reducer that reduces a speed of rotation transmitted from the first speed reducer and activates the joint.

17. The operation table of claim 16, wherein a total reduction ratio of the first and second speed reducers is 1000 or more and 20000 or less.

18. A treatment table comprising:
a table for loading a patient;
a base installed below a floor; and
a robot arm, a first end of the robot arm supported by the base via a slide joint and a second end of the robot arm supporting the table,
wherein the slide joint is configured to slide the robot arm in the vertical direction with respect to the base,
wherein the robot arm comprises:
a vertical joint rotatable about a rotational axis extending in a horizontal direction; and
a joint activation mechanism that activates the vertical joint,
wherein the joint activation mechanism comprises:
a motor;
a first speed reducer that reduces a speed of rotation transmitted from the motor to output slower rotation; and
a second speed reducer that reduces a speed of the slower rotation transmitted from the first speed reducer to output slower rotation.

19. The treatment table of claim 18, wherein the treatment table is a patient positioning device for a radiation treatment system.

20. The treatment table of claim 18, wherein the robot arm comprises a slide joint activation mechanism including:
a second motor; and
a ball screw mechanism or a rack and pinion mechanism.

21. A treatment table comprising:
a table for loading a patient;
a base installed below a floor; and
a robot arm, a first end of the robot arm supported by the base via a slide joint and a second end of the robot arm supporting the table,
wherein the slide joint is configured to slide the robot arm in the vertical direction with respect to the base,
wherein the robot arm comprises:
a plurality of joints; and
a plurality of joint activation mechanisms for the respective joints,
wherein each of the plurality of joint activation mechanisms comprising:
a motor;
a first speed reducer that reduces a speed of rotation transmitted from the motor; and
a second speed reducer that reduces a speed of rotation transmitted from the first speed reducer and activates the joint.
